(19) United States
(12) Patent Application Publication
(10) Pub. No.: US 2011/0281960 A1
(43) Pub. Date: Nov. 17, 2011

(54) METHOD OF SELECTIVELY REMOVING CATALYST FROM FISCHER-TROPSCH SYNTHETIC CRUDE OIL AND METHOD OF RECYCLING REMOVED CATALYST

(76) Inventor: Kazuhiko Tasaka, Yokohama-shi (JP)

(21) Appl. No.: 12/736,106

(22) PCT Filed: Mar. 12, 2009
(86) PCT No.: PCT/JP2009/054779

§ 371 Date: Nov. 5, 2010

(30) Foreign Application Priority Data
Mar. 14, 2008 (JP) 2008 065773
Mar. 14, 2008 (JP) 2008 065778
Mar. 14, 2008 (JP) 2008 065780

(51) Int. Cl. C07C 27/00; C10G 17/00

(52) U.S. Cl. 518/705; 208/251 R

(57) ABSTRACT

A method of selectively removing a Fischer-Tropsch catalyst from a Fischer-Tropsch synthetic crude oil is provided, the method including the steps of extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor; separating a catalyst having a predetermined diameter or more from the slurry by means of a first solid-liquid separator; and separating a catalyst which is not be separated by means of the first solid-liquid separator from the slurry from which the catalyst having the predetermined diameter or more is removed, by means of a second solid-liquid separator.

### TECHNICAL FIELD

The present invention relates to a method of selectively removing a fine catalyst and a low-active catalyst from Fischer-Tropsch synthetic crude oil and a method of recycling a high-active catalyst in Fischer-Tropsch synthetic crude oil.

Priority is claimed on Japanese Patent Application No. 2008-65773, Japanese Patent Application No. 2008-65778, and Japanese Patent Application No. 2008-65780, filed Mar. 14, 2008, the contents of which are incorporated herein by reference.

### BACKGROUND ART

In recent years, a clean liquid fuel containing a low content of sulfur and aromatic hydrocarbons and compatible with the environment has been required from the viewpoint of the reduction of environmental burdens. Therefore, in the oil industry, a Fischer-Tropsch synthesis method (hereinafter, simply referred to as “FT synthesis method”) using carbon monoxide and hydrogen as raw materials has been examined as a method of producing the clean liquid fuel. According to the FT synthesis method, a liquid fuel base stock containing a high content of paraffin and not containing sulfur, for example, a diesel fuel base stock can be produced. For this reason, expectations are high for the FT synthesis method. For example, an environmentally compatible clean liquid fuel is proposed in Patent Document 1.
Patent Document 1: Japanese Patent Application, First Publication No. 2004-323626

Conventionally, an iron-based solid catalyst was widely used as a catalyst for the FT synthesis method using carbon monoxide and hydrogen as raw materials. However, in recent years, a cobalt-based solid catalyst has been developed in view of high activity. Here, in many cases, a reaction type of the FT synthesis method is in a form of a slurry in which solid catalysts are suspended in hydrocarbons of a product. Accordingly, it is necessary not only to obtain FT synthetic crude oil not containing the catalyst, but also to collect the catalyst contained in the slurry so as to reuse the catalyst in view of process cost reduction.

Additionally, a large amount of residual catalyst is contained in the FT synthetic crude oil obtained by the FT synthesis reaction. The FT synthetic crude oil is subjected to upgrading processes such as distillation and hydrotreating processes, thereby obtaining a product such as fuel oil. At this time, since the residual catalyst affects a post-process, for example, the upgrading process, it is necessary to sufficiently remove the residual catalyst from the FT synthetic crude oil.

### DISCLOSURE OF INVENTION

### Technical Problem

Since the slurry having the suspended catalyst is obtained from the FT synthesis reactor, it is preferable not only to obtain the FT synthetic crude oil not containing the catalyst, but also to collect the catalyst for reuse.

However, since the catalyst is changed to fine particles though repeated collision and pulverization occurring in the inside of the FT synthesis reactor, a flowing state of the slurry in the inside of the FT synthesis reactor may be changed.

For this reason, it is necessary to selectively and directly remove the fine catalyst particles so as to maintain fluidity in the FT synthesis reactor and to decrease the catalyst remaining in the FT synthetic crude oil.

Further, in some cases, the catalyst is oxidized by an oxygenated compound in the inside of the FT synthesis reactor. Additionally, in some cases, a coke deposition occurs on the catalyst by an FT synthesis reaction. In any case, activity of the catalyst is decreased and hence efficiency of the catalyst is decreased.

Accordingly, when the catalyst is collected and reused, it is preferable to dispose the low-active catalyst and to collect only the high-active catalyst for reuse.

The present invention is contrived in view of the above-described circumstances, and an object of the invention is to provide a method of selectively removing a fine catalyst and a low-active catalyst from Fischer-Tropsch synthetic crude oil and a method of recycling a high-active catalyst in Fischer-Tropsch synthetic crude oil, according to the concept that a catalyst can be effectively selected based on the value of magnetism of the catalyst because magnetism of the catalyst with a low-activity is weak.

### Technical Solution

According to a first aspect of the invention, there is disclosed a method of selectively removing a catalyst from a Fischer-Tropsch synthetic crude oil, the method including the steps of: extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor; separating a catalyst having a predetermined diameter or more from the slurry by means of a first solid-liquid separator; and separating a catalyst which is not be separated by means of the first solid-liquid separator from the slurry from which the catalyst having the predetermined diameter or more is separated, by means of a second solid-liquid separator.

The catalyst separated from the slurry by means of the first solid-liquid separator is recycled to the Fischer-Tropsch synthesis reactor so as to be reused. The catalyst separated from the slurry by means of the second solid-liquid separator is discharged to the outside of a system. An average particle diameter of the catalyst discharged to the outside of the system is smaller than that of the catalyst contained in the slurry at an outlet of the Fischer-Torpsch synthesis reactor.

In the method of selectively removing the Fischer-Tropsch catalyst according to the first aspect of the invention, the first solid-liquid separator may be a high gradient magnetic separator, and the second solid-liquid separator may be selected as a solid-liquid separator except for the high gradient magnetic separator. Alternatively, the second solid-liquid separator may be a high gradient magnetic separator, and the first solid-liquid separator may be selected as a solid-liquid separator except for the high gradient magnetic separator.

In the method of selectively removing the Fischer-Tropsch catalyst according to the first aspect of the invention, the high gradient magnetic separator may include a washing liquid introduction line for cleaning the inside thereof and a washing liquid discharge line for discharging washing liquid from the high gradient magnetic separator, and may intermittently clean magnetic particles captured in the inside of the high gradient magnetic separator.

In the method of selectively removing the Fischer-Tropsch catalyst according to the first aspect of the invention, the solid-liquid separator except for the high gradient magnetic separator may be at least one of a filter, a gravitational sedimentation separator, a cyclone, and a centrifugal separator.

According to a second aspect of the invention, there is disclosed a method of selectively removing a catalyst from a Fischer-Tropsch synthetic crude oil, the method including the steps of extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor; separating a catalyst having strong magnetism from the slurry by means of a first high gradient magnetic separator; and separating a catalyst which has weak magnetism and is not be separated by means of the first high gradient magnetic separator from the slurry from which the catalyst is separated, by means of a filter.

The catalyst which has strong magnetism and is separated from the slurry by means of the first high gradient magnetic separator is recycled to the Fischer-Tropsch synthesis reactor so as to be reused. The catalyst which has weak magnetism and is separated from the slurry by means of the filter is discharged to the outside of a system.

In the method of selectively removing the Fischer-Tropsch catalyst according to the second aspect of the invention, magnetism of the catalyst separated from the slurry by means of the filter may be weaker than that of the catalyst contained in the slurry at an outlet of the Fischer-Tropsch synthesis reactor.

According to the invention, there is disclosed a method of recycling a Fischer-Tropsch catalyst, the method including the steps of: extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor; separating a catalyst having strong magnetism from the slurry by means of a first high gradient magnetic separator; and separating a catalyst which is not be separated by the first high gradient magnetic separator from the slurry from which the catalyst having strong magnetism is separated, by means of a second high gradient magnetic separator.

The catalyst which has strong magnetism and is separated from the slurry by means of the first high gradient magnetic separator is recycled to the Fischer-Tropsch synthesis reactor so as to be reused. The catalyst separated from the slurry by means of the second high gradient magnetic separator is discharged to the outside of a system.

In the method of recycling the Fischer-Tropsch catalyst according to the invention, magnetism of the catalyst separated from the slurry by means of the first high gradient magnetic separator may be stronger than that of the catalyst contained in the slurry at an outlet of the Fischer-Tropsch synthesis reactor.

### ADVANTAGEOUS EFFECTS

In the method of selectively removing the catalyst from the Fischer-Tropsch synthetic crude oil according to the first aspect of the invention, plural stages of separation steps are provided so as to treat the slurry extracted from the FT synthesis reactor. In the previous stage treatment step, the catalyst having a certain particle diameter is collected from the slurry and is recycled to the FT synthesis reactor so as to be reused. In the downstream step, the fine catalyst is removed so as to obtain the FT synthetic crude oil having a low amount of residual catalyst. Additionally, since the magnetic separation step is also carried out, it is possible to efficiently capture the fine catalyst from the FT synthetic crude oil in which fine microparticles are easily contained.

In the method of selectively removing the catalyst from the Fischer-Tropsch synthetic crude oil according to the second aspect of the invention, since the low-active catalyst is selectively separated and removed from the FT synthetic crude oil and the remainder thereof is recycled, it is possible to improve efficiency of an FT synthesis process.

Additionally, in the separation step using the first high gradient magnetic separator, since most of the residual catalyst having strong magnetism and high activity is separated and removed from the FT synthetic crude oil, most of the remainder is the low-active catalyst. Accordingly, in the separation step using the filter, it is possible to dispose the residual catalyst.

According to the method of recycling the Fischer-Tropsch catalyst, it is possible to obtain the FT synthetic oil having a small amount of residual catalyst and to selectively reuse the residual catalyst having strong magnetism and high activity.

### BRIEF DESCRIPTION OF THE DRAWINGS

FIG. 1 is a schematic view showing a fuel producing plant according to embodiments of the invention.

FIG. 2 is a schematic view showing a high gradient magnetic separator used in the invention.

The reference numeral “10” refers to an FT synthesis reactor; the reference numerals “20, 30” refer to separators; the reference numeral “40” refers to a fractionator.

### BEST MODE FOR CARRYING OUT THE INVENTION

### First Embodiment

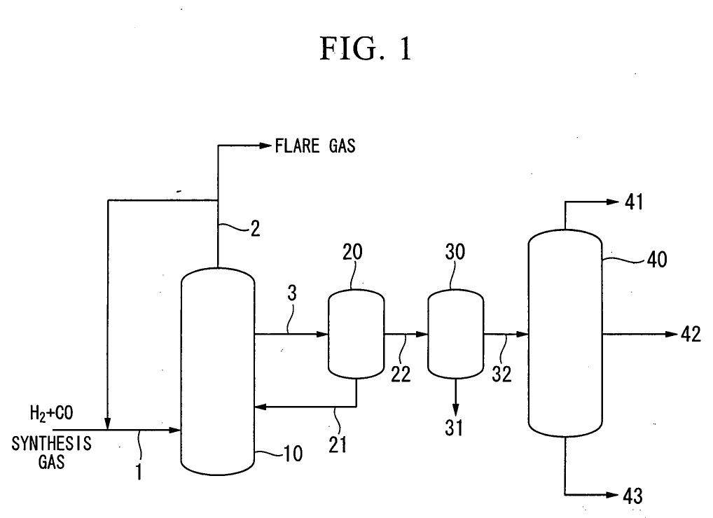

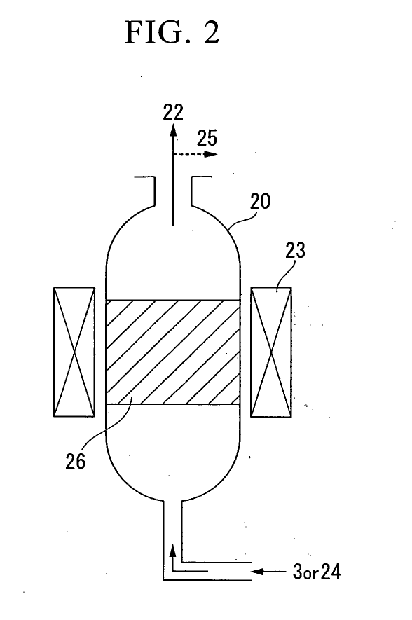

A first embodiment of the invention will be described with reference to FIGS. 1 and 2.

As shown in FIG. 1, synthesis gas containing carbon monoxide gas and hydrogen gas is supplied to an FT synthesis reactor 10 via a line 1 as a synthesis gas supply pipe, thereby producing liquid hydrocarbons by means of an FT synthesis reaction in the FT synthesis reactor 10. The synthesis gas can be obtained, for example, by appropriately reforming hydrocarbon. A typical example of hydrocarbon includes methane, natural gas, LNG (liquid natural gas), and the like. As the reforming method, a partial oxidization reforming method (PDX) using oxygen, an auto thermal reforming method (ATR) that is a combination of the partial oxidation reforming method and a steam reforming method, a carbon dioxide gas reforming method, or the like may be used.

Next, an FT synthesis process will be described with reference to FIG. 1.

An FT synthesis reaction system includes the FT synthesis reactor 10. The FT synthesis reactor 10 is an example of a reactor for obtaining liquid hydrocarbons by synthesizing synthesis gas, and serves as an FT synthesis reactor for synthesizing the liquid hydrocarbons from the synthesis gas by means of an FT synthesis reaction. The reactor 10 may be, for example, a bubble column type reactor.

A reactor body of the FT synthesis reactor 10 is a metallic vessel formed in a substantially cylindrical shape, and has a diameter in the range of from approximately 1 to approximately 20 meters and preferably in the range of from approximately 2 to approximately 10 meters. The reactor body has a height in the range of from approximately 10 to approximately 50 meters and preferably in the range of from approximately 15 to approximately 45 meters. The reactor body contains therein a slurry in which solid catalyst particles are suspended in the liquid hydrocarbons (product of the FT synthesis reaction).

A part of the slurry contained in the FT synthesis reactor 10 is introduced from a body portion of the FT synthesis reactor 10 into a separator 20 via a line 3 as a slurry transfer pipe. Unreacted synthesis gas or the like is discharged from the top of the FT synthesis reactor 10 via a line 2 as a synthesis gas discharge pipe, and a part thereof is recycled to the FT synthesis reactor 10 via the line 1.

The synthesis gas supplied to the FT synthesis reactor 10 via the line 1 is injected from a synthesis gas supply port (not shown) to the slurry contained in the FT synthesis reactor 10. When the synthesis gas comes into contact with the catalyst particles, a synthesis reaction (FT synthesis reaction) of the liquid hydrocarbons occurs due to the contact reaction. Specifically, as shown in the following chemical formula (1), a synthesis reaction occurs between hydrogen gas and carbon monoxide gas.

2nH₂+nCO→CH₂ₙ+nH₂O (1)

Specifically, the synthesis gas is flowed into the bottom of the FT synthesis reactor 10 and moves upward in the slurry contained in the FT synthesis reactor 10. At this time, in the FT synthesis reactor 10, hydrocarbons are produced by the reaction between the hydrogen gas and the carbon monoxide gas contained in the synthesis gas by means of the above-described FT synthesis reaction. Additionally, heat is generated due to the synthesis reaction, but the heat may be removed by means of appropriate cooling means.

An example of a metallic catalyst includes a support type, a precipitation type, and the like, but in any case, the metallic catalyst is a solid magnetic particle containing iron group metal. An appropriate amount of metal is contained in the solid particle, but 100% of the solid particle may be metal. Iron is exemplified as the iron group metal, but cobalt is preferable in view of high activity.

A composition ratio of the synthesis gas supplied to the FT synthesis reactor 10 is set to a composition ratio suitable for the FT synthesis reaction (for example, H₂:CO=2:1 (molar ratio)). Additionally, a pressure of the synthesis gas supplied to the FT synthesis reactor 10 is increased up to a pressure (for example, 3.6 MPaG) suitable for the FT synthesis reaction by means of an appropriate compressor (not shown). However, the compressor may not be provided in some cases.

As described above, the liquid hydrocarbons synthesized by the FT synthesis reactor 10 are extracted from the FT synthesis reactor 10 via the line 3 connected to the body portion of the FT synthesis reactor 10 in a form of a slurry having suspended catalyst particles, and are supplied to a catalyst separation process and a capturing process.

For the catalyst separation process and the capturing process according to the embodiment, as shown in FIG. 1, two separators 20 and 30 are arranged in series.

In a first solid-liquid separation process using the separator 20, a catalyst having a predetermined diameter or more is separated and captured from the slurry. The captured catalyst is recycled to the FT synthesis reactor 10 via a line 21 so as to be reused.

The predetermined diameter can be appropriately set. For example, a particle diameter of the catalyst particle at an initial reaction may be set, or a particle diameter during the reaction while a particle diameter is decreased with time, may be appropriately set with an appropriate time. That is, the diameter of the catalyst required to be separated and collected is appropriately set from the viewpoint of recycling to the reaction system.

Since the catalyst separated and collected by the separator 20 has a large diameter and is reusable by the FT synthesis reactor 10, the catalyst is recycled to the reaction system via the line 21 so as to be reused.

The residual slurry, from which the particles having a large particle diameter are removed, is introduced into the subsequent separator 30 via a line 22.

Since the reusable catalyst is separated and captured by the first solid-liquid separation process, the catalyst remaining in the slurry corresponds to unwanted catalyst having a small particle diameter. The residual catalyst is removed by a second solid-liquid separation, process using the separator 30. Specifically, the catalyst discharged from the separator 30, which has an average particle diameter smaller than that of the catalyst contained in the slurry at the outlet of the FT synthesis reactor 10, is separated. The separated catalyst is discharged to the outside of the system via a line 31. Obtained FT synthetic crude oil can be obtained via a line 32.

Since the catalyst separated and obtained by the separator 30 is in a form of fine powder, the catalyst is discharged to the outside of the system so as to be disposed without reuse.

Either the separators 20 or 30 may be a magnetic separator, but, hereinafter, a case will be described in which the separator 20 is a magnetic separator.

Hereinafter, the first solid-liquid separation process using the high gradient magnetic separator 20 will be described. This process is carried out so as to separate and remove the magnetic particles contained in the extracted FT synthetic crude oil from the FT synthetic crude oil by means of the high gradient magnetic separator 20.

That is, the FT synthetic crude oil is subjected to the magnetic separation process using the high gradient magnetic separator 20 so as to separate and remove the catalyst particles having magnetism. In the type of the iron group metal as the FT synthesis catalyst, it is found that it has a certain magnetic susceptibility and paramagnetism is exhibited regardless of whether it is iron or cobalt. Accordingly, the removal by means of the magnetic separation is effective to a certain degree.

In the high gradient magnetic separator 20 used in the invention, ferromagnetic filling materials are disposed in a uniform high-magnetic-field space formed by an external electromagnetic coil, and ferromagnetic or paramagnetic particles are captured to surfaces of the filling materials by a high magnetic field gradient of 1,000 to 20,000 Gauss/cm in general formed in the periphery of the filling materials, thereby separating the solid magnetic particles such as the catalyst particles from a liquid component containing the liquid hydrocarbons. The filling materials are cleaned, and then the captured particles are removed. As the high gradient magnetic separator 20, for example, a commercially available device known by the trademark as “FEROSEP” and the like may be used.

As the ferromagnetic filling material, a ferromagnetic fine-wire assembly such as steel net or steel wool having a diameter of 1 to 1,000 pin in general, expanded metal, and a conchoidal metallic fine piece may be used. As the metal, it is preferable to use stainless steel having excellent corrosion resistance, heat resistance, and strength.

In addition, the ferromagnetic metal piece proposed in Japanese Patent Application, First Publication No. H07-70568 may be preferably used. That is, the ferromagnetic metal piece is formed into a plate having two planes; a larger-area surface among the two surfaces has the same area as that of a circle having a diameter of R=0.5 to 4.0 mm; a ratio (R/d) between the diameter R and a maximum thickness d of the plate is in the range of 5 to 20; and the plate is made of Fe—Cr base alloy mainly containing Fe and additionally containing Cr of 5 to 25 mass %, Si of 0.5 to 2 mass %, and C of 2 mass % or less.

In the process of separating the magnetic microparticles from the slurry by means of the high gradient magnetic separator 20; the slurry is introduced into the magnetic field space formed by the high gradient magnetic separator 20, and the magnetic particles are captured to the ferromagnetic filling materials disposed in the magnetic field space, thereby removing the magnetic particles from the slurry.

Next, in the process of cleaning, and removing the magnetic particles captured to the filling materials, the ferromagnetic filling materials having the magnetic particles captured thereto are cleaned, thereby removing the magnetic particles from the filling materials by means of washing liquid. The filling materials have a limited surface area used to capture the magnetic particles. Thus, when the magnetic particle capturing amount is equal to or more than a certain amount or a limited amount, the magnetic field is terminated so as to separate the magnetic particles from the filling materials. Subsequently, the filling materials are cleaned by the washing liquid, and then the magnetic particles are discharged to the outside of the magnetic separator together with the washing liquid. A magnetic separation condition for the magnetic particles contained in the slurry and a cleaning and removing condition for the magnetic particles captured to the filling materials will be described hereunder.

As for the magnetic separation condition for the high gradient magnetic separator 20, the magnetic field strength is preferably 2,000 Gauss or more and more preferably 3,000 Gauss or more. A liquid temperature (process temperature) in the magnetic separator is preferably equal to or more than 100° C. and equal to or less than 400° C., more preferably equal to or more than 100° C. and equal to or less than 300° C., and particularly preferably equal to or more than 100° C. and equal to or less than 200° C. A liquid residence time (residence time) is preferably 15 seconds or more and more preferably 20 seconds or more.

Additionally, in the invention, “the liquid residence time” indicates a time obtained in such a manner that a volume of a filling tank applied with a magnetic field is divided by a feed rate of a liquid (i.e., the FT synthetic crude oil fraction containing the magnetic particles) introduced into the filling tank. The liquid residence time is obtained by the following equation.

$$The liquid residence time ( second ) = \frac{\begin{matrix}the volume ( L ) of the filling \\ tank applied with the magnetic field\end{matrix}}{\begin{matrix}the feed rate ( L \text{/} second ) of the FT synthetic \\ crude oil containing the magnetic particles\end{matrix}}$$

Next, when the magnetic separation operation for the magnetic particles is continuously carried out, the removal ratio of the magnetic particles using the filling materials decreases in accordance with an increase in amount of the magnetic particles captured by the filling materials. Accordingly, in order to maintain the removal ratio, it is necessary to carry out the cleaning and removing process for discharging the magnetic particles captured by the filling materials to the outside of the magnetic separator after the magnetic separation operation is continued for a predetermined time. In an industrial operation, liquid containing the magnetic particles may bypass the high gradient magnetic separator during the cleaning and removing process. However, when the time required for the cleaning operation is long, a large amount of the magnetic particles flows into the subsequent process, thereby reducing the removal ratio. Accordingly, a spare switching separator may be provided if necessary.

As the washing liquid used for the cleaning and removing process, the FT synthetic crude oil subjected to the magnetic separation process can be used.

In the cleaning and removing process, the magnetic field formed in the vicinity of the filling materials disappears (the current supply to the magnetic-separation electromagnetic coil stops), and the washing liquid is introduced from the bottom of the separator into the high gradient magnetic separator 20 via a line 24 so as to allow the magnetic particles captured to the filling materials to be flowed outside together with the washing liquid. The washing liquid is discharged to the outside of the system via a line 25. As the cleaning condition, a cleaning-liquid linear velocity is in the range of 1 to 10 cm/sec and preferably in the range of 2 to 6 cm/sec.

Hereinafter, the magnetic separation process will be described with reference to FIG. 2.

FIG. 2 is a schematic view showing the high gradient magnetic separator 20 used in the invention. A separation portion of the high gradient magnetic separator 20 is formed into a vertical filling tower which is filled with the ferromagnetic filling materials. A filling tank 26 filled with the filling materials is magnetized by the lines of magnetic force formed by an electromagnetic coil 23 disposed on the outside of the vertical filling tower to thereby form a high gradient magnetic separation portion. This portion corresponds to the uniform high-magnetic-field space formed by the external electromagnetic coil 23. The slurry heated up to a temperature suitable for the operation is introduced into the bottom of the high gradient magnetic separator 20 via the line 3, and passes through the high gradient magnetic separator 20 from the downside to the upside at a predetermined feed rate (preferably, a feed rate at which the liquid residence time in the magnetic separation portion is 15 seconds or more), thereby discharging the slurry from the top of the high gradient magnetic separator 20 via the line 22. At this time, the magnetic particles contained in the slurry are captured to the surfaces of the filling materials during a time when the slurry passes through the magnetic separation portion.

During a time when the FT synthetic crude oil passes through the high gradient magnetic separator 20, the washing liquid bypasses the high gradient magnetic separator 20 via a washing liquid bypass line (not shown). During a time when the filling materials having the magnetic particles captured thereto are cleaned, the washing liquid is introduced into the high gradient magnetic separator 20 via the line 24. The slurry may bypass the high gradient magnetic separator 20 via a slurry bypass line (not shown) or may be transferred to the solid-liquid separator at the downstream, for example, the separator 30. In this manner, it is possible to carry out the switching operation of the removing operation and the cleaning operation, and the repeated continuous operation. The cleaning and removing process can be carried out on the basis of, for example, the method disclosed in Japanese Patent Application, First Publication No. H06-200260.

Next, the second solid-liquid separation process using the separator 30 except for the magnetic separator will be described.

A liquid component, from which the catalyst particles are separated by the first solid-liquid separation process, is introduced into the separator 30 via the line 22. In the separator 30, the catalyst is further separated from the liquid component separated by the first solid-liquid separation process. An average particle diameter of the catalyst is smaller than an average particle diameter of the catalyst contained in the slurry at the outlet of the reactor 10. The separated catalyst is discharged to the outside of the system via the line 31. The clean FT synthetic crude oil, in which the residual catalyst is reduced, is extracted from the separator 30 via the line 32, and is introduced into a subsequent process, for example, a fractionator 40.

A method of measuring an average particle diameter is not particularly limited. For example, it is preferable to use an average particle diameter (μm) measured by a particle size distribution analyzer using a laser diffraction method, a dynamic light scattering method, or a standard sieve method. Additionally, an average particle diameter of the disposed FT catalyst is not particularly limited so long as the average particle diameter is smaller than the average particle diameter of the FT catalyst contained in the slurry at the outlet of the FT synthesis reactor 10. The average particle diameter of the disposed FT catalyst is smaller than the average particle diameter of the FT catalyst contained in the slurry at the outlet of the FT synthesis reactor by preferably 5% or more, more preferably 10% or more, and still more preferably 20% or more. A lower limit value is not particularly limited. In general, the lower limit value is dependent on a separation performance at the second-stage solid-liquid separation process.

In the second solid-liquid separation process using the separation means except for the magnetic separator at the downstream, the separation means may be configured as a known separator. For example, a filter using an appropriate filter element such as a sintered metallic filter element, a gravitational sedimentation separator, a cyclone, a centrifugal separator, or the like is adopted. For example, as a gravitational sedimentation separator, a sedimentation tank (a gravitational sedimentation separator) may be used which is filled with a liquid component for a predetermined time so that solid particles contained in the liquid component are spontaneously settled out. The gravitational sedimentation separator is advantageous due to a simple structure. All of a continuous type or a batch type may be used. As this embodiment, it is more preferable to use a filter type separator having a filter element with an appropriate mesh opening size, such as a sintered metallic filter element, from the viewpoint that it is easy to separate catalyst particles.

In, the above description, as the first-stage separator 20, the magnetic separator is selected, and as the second-stage separator 30, the separation means except for the magnetic separator is selected. However, the invention is not limited thereto. On the contrary, as the first-stage separator 20, the separation means except for the magnetic separator may be selected and as the second-stage separator 30, the magnetic separator may be selected.

As shown in FIG. 1, the FT synthetic crude oil, from which the magnetic particles are removed by the separators 20 and 30, is introduced, into the fractionator 40 via the line 32 so as to be fractionally distilled therein and is subjected to various upgrading processes such as the hydrotreating process, thereby obtaining a product.

That is, the FT synthetic crude oil obtained by the two stages of solid-liquid separation processes is introduced into the fractionator 40 so as to be fractionally distilled therein. Additionally, for example, a naphtha fraction (having a boiling point of approximately less than 150° C.) is fractionally distilled via a line 41, a middle fraction (having a boiling point in the range of from approximately 150° C. to approximately 350° C.) is fractionally distilled via a line 42, and then a wax fraction (having a boiling point of approximately more than 350° C.) is fractionally distilled via a line 43. Further, in FIG. 1, three fractions are obtained by the fractional distillation, but two fractions may be obtained or three or more fractions may be obtained by the fractional distillation. Furthermore, the FT synthetic crude oil may be supplied to the subsequent upgrading process without a particular fractional distillation.

### Example 1

Synthesis gas obtained by reforming natural gas and mainly containing carbon monoxide and hydrogen gas is introduced into the hydrocarbon synthesis reactor (FT synthesis reactor) 10 of a bubble column type via the line 1 so as to induce a reaction with a slurry having suspended FT catalyst particles (having an average particle diameter of 100 μm and cobalt loaded as active metal of 30 mass %), thereby synthesizing liquid hydrocarbons.

The liquid hydrocarbons synthesized in the FT synthesis reactor 10 are extracted from the FT synthesis reactor 10 via the line 3 in a form of a slurry containing FT catalyst particles.

The extracted slurry is introduced to the electromagnetic high gradient magnetic separator 20 (FEROSEP (trademark)) provided for the first solid-liquid separation process disposed at the downstream of the FT synthesis reactor so as to be separated into a particle having a comparatively large particle diameter and a liquid component (liquid A) under the process condition marked in TABLE 1.

The catalyst particles separated by the first solid-liquid separation process are recycled to the FT synthesis reactor 10 via the line 21. The liquid component (liquid A) containing the catalyst particles, which are not be captured by the high gradient magnetic separator 20, is introduced to the separator 30 (sintered metallic filter element having a mesh opening size of 10 μm) provided for the second solid-liquid separation process via the line 22, thereby the liquid component is divided into a liquid component (liquid B) and a solid component (a catalyst particle).

Additionally, the high gradient magnetic separator 20 used in the first solid-liquid separation process includes a washing liquid introduction line 24 for cleaning the inside of the high gradient magnetic separator 20 and a cleaning liquid discharge line 25 for discharging the washing liquid. The high gradient magnetic separator 20 intermittently cleans the catalyst particles separated from the FT synthetic crude oil every two hours and recycles the cleaned catalyst particles to the FT synthesis reactor 10.

The catalyst particles separated by the second solid-liquid separation process are discharged to the outside of the system. The liquid component (liquid B) is introduced to the fractionator 40 so as to obtain the naphtha fraction (having a boiling point of approximately less than 150° C.) fractionally distilled via the line 41, the middle fraction (having a boiling point in the range of from approximately 150° C. to approximately 350° C.) fractionally distilled via the line 42, and the wax fraction (having a boiling point of approximately more than 350° C.) fractionally distilled via the line 43. Additionally, the middle fraction is subjected to a process of a hydroisomerizing device (not shown) and the wax fraction is subjected to a process of a hydrocracking device (not shown). Subsequently, the effluents from hydroisomerizing device and hydrocracking device are mixed in the line and are introduced into a second fractionator (not shown) for a fractional distillation therein, thereby obtaining a diesel fuel base stock.

At this time, the average particle diameter of the catalyst particles contained in the slurry at the outlet of the FT synthesis reactor 10 is 72.5 μm; and the average particle diameter of the catalyst particles discharged to the outside of the system is 57 μm.

Additionally, the average particle diameter of the catalyst particles is a value measured by means of a laser diffraction particle size distribution analyzer (SALD-3100) manufactured by SHIMADZU Corporation (hereinafter, the same applies).

### Example 2

The same process is carried out as that of Example 1 except that the process condition for the first solid-liquid separation process is changed to the value marked in TABLE 1. At this time, the average particle diameter of the catalyst particles discharged to the outside of the system is 28 μm.

### Example 3

The same process is carried out as that of Example 1 except that the process condition for the first solid-liquid separation process is changed to the value marked in TABLE 1. At this time, the average particle diameter of the catalyst particles discharged to the outside of the system is 39 μm.

### Example 4

The same process is carried out as that of Example 1 except that the process condition for the first solid-liquid separation process is changed to the value marked in TABLE 1. At this time, the average particle diameter of the catalyst particles discharged to the outside of the system is 25 μm.

### Example 5

Synthesis gas obtained by reforming natural gas and mainly containing carbon monoxide and hydrogen gas is introduced into the hydrocarbon synthesis reactor (FT synthesis reactor) 10 of a bubble column type via the line 1 so as to induce a reaction with a slurry having suspended FT catalyst particles (having an average particle diameter of 100 μm and cobalt loaded as active metal of 30 mass %), thereby synthesizing liquid hydrocarbons.

The liquid hydrocarbons synthesized in the FT synthesis reactor 10 are extracted from the FT synthesis reactor 10 via the line 3 in a form of a slurry containing FT catalyst particles.

The extracted slurry is introduced to the separator 20 (sintered metallic filter element having a mesh opening size of 10 μm) provided for the first solid-liquid separation process disposed at the downstream of the FT synthesis reactor 10 so as to be divided into a particle having a comparatively large particle diameter and a liquid component (liquid A).

The catalyst particles separated by the first solid-liquid separation process are recycled to the FT synthesis reactor 10 via the line 21. The liquid component (liquid A) containing the catalyst particles, which is not be captured by the separator 20 such as the sintered metallic filter element, is introduced to the electromagnetic high gradient magnetic separator 30 (FEROSEP (trademark)) provided for the second solid-liquid separation process so as to be divided into a liquid component (liquid B) and a catalyst particle which is a solid component.

Additionally, the high gradient magnetic separator 30 used in the second solid-liquid separation process includes the washing liquid introduction line 24 for cleaning the inside of the high gradient magnetic separator 30 and the washing liquid discharge line 25 for discharging the washing liquid. The high gradient magnetic separator 30 intermittently cleans the captured catalyst particles every two hour and discharges the cleaned catalyst particles to the outside of the system.

The catalyst particles separated by the second solid-liquid separation process are discharged to the outside of the system. The liquid component (liquid B) is introduced to the fractionator 40 to thereby obtain the naphtha fraction (having a boiling point of approximately less than 150° C.) fractionally distilled via the line 41, the middle fraction (having a boiling point in the range of from approximately 150° C. to approximately 350° C.) fractionally distilled via the line 42, and the wax fraction (having a boiling point of approximately more than 350° C.) fractionally distilled via the line 43. Additionally, the middle fraction is subjected to the process of the hydroisomerizing device (not shown), and the wax fraction is subjected to the process of the hydrocracking device (not shown). Subsequently, the effluents from hydroisomerizing device and hydrocracking device are mixed in the line and are introduced into the second fractionator (not shown) for a fractional distillation therein, thereby obtaining the diesel fuel base stock.

At this time, the average particle diameter of the catalyst particles contained in the slurry at the outlet of the FT synthesis reactor 10 is 72.5 μm, and the average particle diameter of the catalyst particles discharged to the outside of the system is 25 μm.

### Comparative Example 1

Synthesis gas obtained by reforming natural gas and mainly containing carbon monoxide and hydrogen gas is introduced into the hydrocarbon synthesis reactor (FT synthesis reactor) 10 of a bubble column type via the dine 1 so as to induce a reaction with slurry having suspended FT catalyst particles (having an average particle diameter of 100 μm and cobalt loaded as active metal of 30 mass %), thereby synthesizing liquid hydrocarbons.

The liquid hydrocarbons synthesized in the FT synthesis reactor 10 are extracted from the FT synthesis reactor 10 via the line 3 in a form of a slurry containing FT catalyst particles.

The extracted slurry is introduced to the separator 20 (sintered metallic filter element having a mesh opening size of 10 μm) provided for the solid-liquid separation process disposed at the downstream of the FT synthesis reactor so as to be divided into a catalyst particle and a liquid component. Here, the solid-liquid separator 20 is provided for a single stage, and the magnetic separator 30 is not provided.

The catalyst particles separated by the single-stage solid-liquid separator 20 are discharged to the outside of the system. The liquid component is introduced to the fractionator 40. Subsequently, the naptha fraction (having a boiling point of approximately less than 150° C.) is fractionally distilled via the line 41, the middle fraction (having a boiling point in the range of from approximately 150° C. to approximately 350° C.) is fractionally distilled via the line 42, and then the wax fraction (having a boiling point of approximately more than 350° C.) is fractionally distilled via the line 43. At this time, the average particle diameter of the catalyst particles discharged to the outside of the system is 72.5 μm.

TABLE 1

| | | EXAMPLE | EXAMPLE | EXAMPLE | EXAMPLE | EXAMPLE | COMPARATIVE |
|---|---|---|---|---|---|---|---|
| | | 1 | 2 | 3 | 4 | 5 | EXAMPLE 1 |
| FIRST-STAGE SOLID-LIQUID SEPARATOR | | FEROSEP | FEROSEP | FEROSEP | FEROSEP | SINTERED | NO |
| (MAGNETIC SEPARATION) | | | | | | METALLIC | |
| | | | | | | FILTER | |
| | | | | | | ELEMENT: | |
| | | | | | | MESH | |
| | | | | | | OPENING | |
| | | | | | | SIZE OF 10 | |
| | | | | | | μm | |
| SECOND-STAGE SOLID-LIQUID | | SINTERED | SINTERED | SINTERED | SINTERED | FEROSEP | SINTERED |
| SEPARATOR | | METALLIC | METALLIC | METALLIC | METALLIC | | METALLIC |
| | | FILTER | FILTER | FILTER | FILTER | | FILTER |
| | | ELEMENT: | ELEMENT: | ELEMENT: | ELEMENT: | | ELEMENT: |
| | | MESH | MESH | MESH | MESH | | MESH |
| | | OPENING | OPENING | OPENING | OPENING | | OPENING |
| | | SIZE OF 10 | SIZE OF 10 | SIZE OF 10 | SIZE OF 10 | | SIZE OF 10 |
| | | μm | μm | μm | μm | | μm |
| PROCESS | MAGNETIC FIELD | 3000 | 3000 | 2000 | 4000 | 3000 | — |
| CONDITION FOR | STRENGTH (Gauss) | | | | | | |
| MAGNETIC | PROCESS | 150 | 150 | 150 | 150 | 150 | — |
| SEPARATOR | TEMPERATURE (° C.) | | | | | | |
| | LIQUID RESIDENCE | 20 | 50 | 50 | 50 | 20 | — |
| | TIME (SECOND) | | | | | | |

| | | | | | | |
|---|---|---|---|---|---|---|
| AVERAGE PARTICLE DIAMETER (BASED | 57 | 28 | 39 | 25 | 25 | 72.5 |
| ON WEIGHT) OF DISCHARGED CATALYST | | | | | | |
| μm | | | | | | |

(Results)

In the case where the electromagnetic high gradient magnetic separator 20 used for the first solid-liquid separation process and the separator 30 such as a metallic filter element used for the second solid-liquid separation process are disposed at the downstream of the FT synthesis reactor (Examples 1 to 4), and the case where the separator 20 such as the metallic filter element used for the first solid-liquid separation process and the electromagnetic high gradient magnetic separator 30 used for the second solid-liquid separation process are disposed at the downstream of the FT synthesis reactor, in any case, the average particle diameter (based on the weight) of the catalyst discharged to the outside of the system has a value smaller than that shown in Comparative Example 1. That is, it is understood that the catalyst particles of which the particle diameter is reduced, are selectively removed from the slurry.

### Second Embodiment

A second embodiment of the invention will be described with reference to FIGS. 1 and 2.

For the catalyst separation process and the capturing process according to the embodiment, as shown in FIG. 1, two separators 20 and 30 are arranged in series. In the first solid-liquid separation process, a high gradient magnetic separator is used as the separator 20. In the second solid-liquid separation process, a filter is used as the separator 30.

That is, the high gradient magnetic separator 20 separates the catalyst particles having strong magnetism. Since the catalyst particles having strong magnetism are particles still having high reaction activity, the catalyst particles are recycled to the FT synthesis reactor 10 via the line 21 so as to be reused. The magnetism of particles to be recycled to the FT synthesis reactor 10 may be arbitrarily set. For example, the slurry is extracted from the FT (synthesis reactor 10 before being subjected to the high gradient magnetic separator 20. Then, the particles having magnetism stronger than that of the catalyst particle in the extracted slurry, are captured and separated so as to be recycled.

The slurry from which the particles having strong magnetism are removed, is supplied to the second solid-liquid separation process using the filter 30 via the line 22.

Since the first solid-liquid separation process using the high gradient magnetic separator 20 is described already in the first embodiment, the description thereof will be omitted. However, as for the separation condition for the high gradient magnetic separator 20 according to the embodiment, the magnetic field strength is preferably 15,000 Gauss or more and more preferably 30,000 Gauss or more. The liquid temperature (process temperature) in the magnetic separator is preferably equal to or more than 100° C. and equal to or less than 400° C., more preferably equal to or more than 100° C. and equal to or less than 300° C., and particularly preferably equal to or more than 100° C. and equal to or less than 200° C. The liquid residence time is preferably 15 seconds or more and more preferably 50 seconds or more.

In the embodiment, the high gradient magnetic separator 20 is capable of separating the magnetic particles by appropriately setting the separation condition.

Next, the second solid-liquid separation process using the filter 30 will be described, where the filter using an appropriate filter element such as a sintered metallic filter element, is selected.

The liquid component, from which the catalyst particles are separated by the first solid-liquid separation process, is introduced into the filter 30 via the line 22. In the second solid-liquid separation process, the filter 30 may be selected as a known separator. For example, a gravitational sedimentation separator, a cyclone, a centrifugal separator, or the like is adopted in addition to the filter. For example, as a gravitational sedimentation separator, a sedimentation tank (a gravitational sedimentation separator) may be used which is filled with a liquid component for a predetermined time so that solid particles contained in the liquid component are spontaneously settled out. The gravitational sedimentation separator is advantageous due to a simple structure. All of a continuous type or a batch type may be used.

Since the high-active particles having strong magnetism are already captured by the first solid-liquid separation process, and the particles having weak magnetism, that is, low-active catalyst particles are introduced into the filter 30 used for the second solid-liquid separation process, a filter element having a minute mesh opening size is used. Then, the separated catalyst is discharged to the outside of the system without recycle, and is preferably disposed. That is, the catalyst particles discharged to the outside of the system in the second solid-liquid separation process have magnetism smaller than that of the FT catalyst contained in the slurry at the outlet of the FT synthesis reactor 10. Accordingly, such catalyst particles are discharged to the outside of the system via the line 31.

A method of measuring the magnetism is not particularly limited, but magnetic susceptibility (emu/g) can be preferably measured by, for example, a SQUID (superconducting quantum interference device) or the like. Additionally, the magnetism of the disposed catalyst particles is not particularly limited so long as the magnetism is smaller than that of the FT catalyst contained in the slurry at the outlet of the FT synthesis reactor 10. Concerning the value of magnetic susceptibility, the magnetic susceptibility of the disposed catalyst particles may be equal or less than 98% of than that of the FT catalyst contained in the slurry at the outlet of the FT synthesis reactor 10, preferably is equal or less than 97% of than that of the FT catalyst.

Since the clean FT synthetic crude oil, in which the residual catalyst is reduced, is obtained by the known filter operation described above, it is possible to selectively remove the FT catalyst having small magnetism and low activity.

The FT synthetic crude oil, from which the magnetic particles are separated by the separators 20 and 30, is introduced into the fractionator 40 via the line 32 as shown in FIG. 1 for a fractional distillation therein, and is subjected to various upgrading processes such as a hydrotreating process, thereby obtaining a product.

### Example 6

Synthesis gas obtained by reforming natural gas and mainly containing carbon monoxide and hydrogen gas is introduced into the hydrocarbon synthesis reactor (FT synthesis reactor) 10 of a bubble column type via the line 1 so as to induce a reaction with slurry having suspended FT catalyst particles (having an average particle diameter of 100 μm and cobalt loaded as active metal of 30 mass %), thereby synthesizing liquid hydrocarbons.

The liquid hydrocarbons synthesized in the FT synthesis reactor 10 are extracted from the FT synthesis reactor 10 via the line 3 in a form of slurry containing FT catalyst particles.

The extracted slurry is introduced to the electromagnetic high gradient magnetic separator 20 (FEROSEP (trademark)) provided for the first solid-liquid separation process disposed at the downstream of the FT synthesis reactor 10 so as to be divided into a part of catalyst particle and a liquid component (liquid A) under the process condition marked in TABLE 2.

The catalyst particles separated by the first solid-liquid separation process are recycled to the FT synthesis reactor 10 via the line 21. Then, the liquid component (liquid A) containing the catalyst particles, which is not be captured by the high gradient magnetic separator 20, is introduced to the filter 30 (sintered metallic filter element having a mesh opening size of 10 μm) provided for the second solid-liquid separation process via the line 22, thereby being divided into a liquid component (liquid B) and a catalyst particle which is a solid component.

The catalyst particles separated by the second solid-liquid separation process are discharged to the outside of the system. The liquid component (liquid B) is introduced to the fractionator 40 to thereby obtain the naphtha fraction (having a boiling point of approximately less than 150° C.) fractionally distilled via the line 41, the middle fraction (having a boiling point in the range of from approximately 150° C. to approximately 350° C.) fractionally distilled via the line 42, and the wax fraction (having a boiling point of approximately more than 350° C.) fractionally distilled via the line 43. Additionally, the middle fraction is subjected to a process of a hydroisomerizing device (not shown) and the wax fraction is subjected to a process of a hydrocracking device (not shown). Subsequently, the effluents from hydroisomerizing device and hydrocracking device are mixed in the line and are introduced into a second fractionator (not shown) for a fractional distillation therein, thereby obtaining the diesel fuel base stock.

At this time, the magnetic susceptibility of the catalyst particles contained in the slurry at the outlet of the FT synthesis reactor 10 is 7.30 emu/g, and the magnetic susceptibility of the catalyst particles discharged to the outside of the system is 7.13 emu/g.

Additionally, the magnetic susceptibility of the catalyst particles is a value measured by a SQUID (superconducting quantum interference device) magnetic flux meter (MPMS-5 manufactured by Quantum Design, Inc.) (hereinafter, the same applies).

### Examples 7 to 9

The same process as that of Example 6 is carried out except that the process condition for the high gradient magnetic separator 20 is changed to the value marked in TABLE 2. Each average magnetic susceptibility of the disposed FT catalyst particles according to the Examples is marked in TABLE 2.

### Comparative Example 2

The same process as that of Example 6 is carried out except that the high gradient magnetic separator 20 is not used for the process of separating the FT catalyst particles from the slurry. The magnetic susceptibility of the disposed FT catalyst particles according to Comparative Example 2 is marked in TABLE 2.

TABLE 2

| | | EXAMPLE | EXAMPLE | EXAMPLE | EXAMPLE | COMPARATIVE |
|---|---|---|---|---|---|---|
| | | 6 | 7 | 8 | 9 | EXAMPLE 2 |
| FIRST-STAGE HIGH GRADIENT | | FEROSEP | FEROSEP | FEROSEP | FEROSEP | — |
| MAGNETIC SEPARATOR | | | | | | |
| PROCESS | MAGNETIC FIELD | 15000 | 30000 | 15000 | 30000 | — |
| CONDITION FOR | STRENGTH (Gauss) | | | | | |
| FIRST-STAGE | PROCESS | 150 | 150 | 150 | 150 | — |
| MAGNETIC | TEMPERATURE (° C.) | | | | | |
| SEPARATION | LIQUID RESIDENCE | 50 | 50 | 100 | 100 | — |
| | TIME (SECOND) | | | | | |

| | | | | | |
|---|---|---|---|---|---|
| SECOND-STAGE FILTER | FILTER | FILTER | FILTER | FILTER | FILTER |
| | ELEMENT | ELEMENT | ELEMENT | ELEMENT | ELEMENT |
| | MESH | MESH | MESH | MESH | MESH |
| | OPENING | OPENING | OPENING | OPENING | OPENING |
| | SIZE: 10 μm | SIZE: 10 μm | SIZE: 10 μm | SIZE: 10 μm | SIZE: 10 μm |
| MAGNETIC SUSCEPTIBILITY (emu/g) OF | 7.13 | 7.01 | 7.01 | 6.75 | 7.25 |
| DISPOSED FT CATALYST | | | | | |

(Result)

It is understood that the FT catalyst particles discharged to the outside of the system by the separation processes using the high gradient magnetic separator 20 and the filter 30 have magnetism and activity smaller than those of the FT catalyst particles contained in the slurry at the outlet of the FT synthesis reactor 10. On the other hand, in the case where the FT catalyst particles are disposed by using only the filter 30, the catalyst having comparatively strong magnetism is also disposed.

### Third Embodiment

A third embodiment of the invention will be described with reference to FIGS. 1 and 2.

For the catalyst separation process and the capturing process according to the embodiment, as shown in FIG. 1, two separators 20 and 30 are arranged in series. In the embodiment, a high gradient magnetic separator is used as each of the separators 20 and 30. In the first high gradient magnetic separator 20 and the second high gradient magnetic separator 30, both operation conditions are set to different values so that the magnetisms of the catalyst particles separated and captured by the respective separators are different from each other.

That is, the first high gradient magnetic separator 20 separates the catalyst particles having strong magnetism. Since the catalyst particles having strong magnetism are particles having high reaction activity, the catalyst particles are recycled to the FT synthesis reactor 10 via the line 21 so as to be reused. The magnetism of particles to be recycled to the FT synthesis reactor 10 may be arbitrarily set. For example, the magnetism of the catalyst particles removed from the slurry by using the high gradient magnetic separator 20 and recycled to the FT synthesis reactor 10 can be set to be larger than that of the FT catalyst contained in the slurry at the outlet of the FT synthesis reactor 10. Accordingly, it is possible to selectively recycle only the FT catalyst having strong magnetism and high activity to the FT synthesis reactor.

A method of measuring the magnetism is not particularly limited, but magnetic susceptibility (emu/g) can be measured by, for example, a SQUID (superconducting quantum interference device) or the like. Additionally, the magnetism of the catalyst particles recycled to the FT synthesis reactor is not particularly limited so long as the magnetism is larger than that of the FT catalyst contained in the slurry at the outlet of the FT synthesis reactor 10. Concerning the value of magnetic susceptibility, the magnetic susceptibility of the recycled catalyst particles is larger than that of the FT catalyst contained in the slurry at the outlet of the FT synthesis reactor 10 by preferably 0.5% or more or 1.0% or more.

Since the catalyst separated and collected by the process of the first high gradient magnetic separator 20 still has high activity as described above, the catalyst is recycled to the FT synthesis reactor 10 via the line 21 so as to be reused.

The slurry from which the strong magnetic particles are removed, is supplied to the second solid-liquid separation process using the second high gradient magnetic separator 30 via the line 22.

Since the catalyst separated and captured from the slurry by the high gradient magnetic separator 30 has weak magnetism and low activity, the catalyst is discharged to the outside of the system via the line 31.

Since the first solid-liquid separation process using the high gradient magnetic separator 20 is described already in the first embodiment, the description thereof will be omitted. However, as for the separation condition fore the high gradient magnetic separator 20 according to the embodiment, the magnetic field strength is preferably 5,000 Gauss or more and more preferably 15,000 Gauss or more. The liquid temperature (process temperature) in the magnetic separator is preferably equal to or more than 100° C. and equal to or less than 400° C., more preferably equal to or more than 100° C. and equal to or less than 300° C., and particularly preferably equal to or more than 100° C. and equal to or less than 200° C. The liquid residence time is preferably 10 seconds or more and more preferably 50 seconds or more.

In the embodiment, the high gradient magnetic separator 20 is capable of separating the magnetic particles by appropriately setting the separation condition. For example, the magnetism of the FT catalyst removed from the slurry by the high gradient magnetic separator 20 and recycled to the FT synthesis reactor can be set to be larger than that of the FT catalyst contained in the slurry at the outlet of the FT synthesis reactor 10.

Next, the second solid-liquid separation process using the high gradient magnetic separator 30 will be described. Additionally, the structure and the operation of the high gradient magnetic separator 30 are the same as those of the high gradient magnetic separator 20. However, the high gradient magnetic separator 30 magnetically separates the particles having weak magnetism. As described above, since the catalyst particles having weak magnetism are already separated and removed by the high gradient magnetic separator 20, the catalyst remaining in the FT synthetic crude oil introduced into the high gradient magnetic separator 30 has weak magnetism and low activity. Such catalyst particles having weak magnetism and low activity need to be separated by the high gradient magnetic separator 30 as much as possible so as to be discharged to the outside of the system.

As for the separation condition for the high gradient magnetic separator 30, the magnetic field strength is preferably 15,000 Gauss or more and more preferably 20,000 Gauss or more. The liquid temperature (process temperature) in the magnetic separator is preferably equal to or more than 100° C. and equal to or less than 400° C., more preferably equal to or more than 100° C. and equal to or less than 300° C., and particularly preferably equal to or more than 100° C. and equal to or less than 200° C. The liquid residence time is preferably 50 seconds or more.

The catalyst particles separated and removed by the high gradient magnetic separator 30 have weak magnetism and low activity. Accordingly, such catalyst particles are not recycled to the FT synthesis reactor 10, but are discharged to the outside of the system via the line 31. Preferably, such catalyst particles are disposed.

The FT synthetic crude oil, from which the magnetic particles are separated, is introduced into the fractionator 40 via the line 32.

Additionally, in the high gradient magnetic separator 30 used for the second solid-liquid separation process, it is possible to remove a large amount of residual catalyst by, appropriately controlling the operation condition. Accordingly, it is possible to obtain the FT synthetic crude oil from which the residual catalyst is removed.

The FT synthetic crude oil, from which the magnetic particles are separated by the separators 20 and 30, is introduced into the fractionator 40 via the line 32 for a fractional distillation therein as shown in FIG. 1, and is subjected to various upgrading processes such as the hydrotreating process, thereby obtaining a product.

### Example 10

Synthesis gas obtained by reforming natural gas and mainly containing carbon monoxide and hydrogen gas is introduced into the hydrocarbon synthesis reactor (FT synthesis reactor) 10 of a bubble column type via the line 1 so as to induce a reaction with slurry having suspended FT catalyst particles (having an average particle diameter of 100 μm and cobalt loaded as active metal of 30 mass %), thereby synthesizing liquid hydrocarbons.

The liquid hydrocarbons synthesized in the FT synthesis reactor 10 are extracted from the FT synthesis reactor 10 via the line 3 in a form of slurry containing FT catalyst particles.

The extracted slurry (FT catalyst concentration: 100 mass ppm) is introduced to the first electromagnetic high gradient magnetic separator 20 (FEROSEP (trademark)) provided for the first solid-liquid separation process disposed at the downstream of the FT synthesis reactor 10 so as to be divided into a part of catalyst particle (having magnetism, stronger than that of the FT catalyst particles contained in the slurry at the outlet of the FT synthesis reactor 10) and a liquid component (liquid A) under the process condition marked in TABLE 3.

The high active catalyst particles separated by the first solid-liquid separation process are recycled to the FT synthesis reactor 10 via the line 21. Then, the liquid component (liquid A) containing the catalyst particles, which is not captured by the high gradient magnetic separator 20, is introduced to the second high gradient magnetic separator 30 provided for the second solid-liquid separation process via the line 22 so as to be divided into an FT catalyst particle, which is a solid component, and a liquid component (liquid B) under the process condition marked in TABLE 3.

The low-active catalyst particles separated by the second solid-liquid separation process are discharged to the outside of the system via the line 31. Then, the liquid component (liquid B) is introduced to the fractionator 40 so as to obtain the naphtha fraction (having a boiling point of approximately less than 150° C.) fractionally distilled via the line 41, the middle fraction (having a boiling point in the range of from approximately 150° C. to approximately 350° C.) fractionally distilled via the line 42, and the wax fraction (having a boiling point of approximately more than 350° C.) fractionally distilled via the line 43. Additionally, the middle fraction is subjected to a process of a hydroisomerizing device (not shown) and the wax fraction is subjected to a process of a hydrocracking device (not shown). Subsequently, the effluents from hydroisomerizing device and hydrocracking device are mixed in the line and are introduced into a second fractionator (not shown) for a fractional distillation therein, thereby obtaining the diesel fuel base stock.

At this time, the magnetic susceptibility of the catalyst particles contained in the slurry at the outlet of the FT synthesis reactor 10 is 7.30 emu/g, and the magnetic susceptibility of the FT catalyst particles separated and removed by the high gradient magnetic separator 20 and recycled to the FT synthesis reactor 10 via the line 21 is 7.36 emu/g.

The catalyst concentration of the catalyst particles of liquid B at the outlet of the high gradient magnetic separator 30 is 9.6 mass ppm.

Additionally, the magnetic susceptibility of the catalyst particles is a value measured by a SQUID (superconducting quantum interference device) magnetic flux meter (MPMS-5 manufactured by Quantum Design, Inc.) (hereinafter, the same applies).

Further, the catalyst concentration (mass ppm) of the catalyst particle of liquid B is a value calculated based on the weight of the processed oil and the measurement result obtained by the laser diffraction particle size distribution analyzer (SALD-3100) manufactured by SHIMADZU Corporation.

### Examples 11 to 14

The same process is carried out as that of Example 10 except that the process conditions of the first high gradient magnetic separator 20 and the second high gradient magnetic separator 30 are changed to the values shown in TABLE 3. Each magnetic susceptibility of the recycled FT catalyst particles according to the respective Examples and each catalyst concentration at the outlet of the second high gradient magnetic separator 30 is marked in TABLE 3.

### Comparative Example 3

The same process is carried out as that of Example 10 except that the sintered metallic filter element having a mesh opening size of 10 μm is used instead of the first high gradient magnetic separator 20 and the second high gradient magnetic separator 30 in the process of separating the FT catalyst particle from the slurry. The magnetic susceptibility of the recycled FT catalyst particles and the catalyst concentration of the processed oil at the outlet of the filter are marked in TABLE 3. Additionally, the catalyst concentration of the processed oil at the outlet of the filter is marked in an edit box of the catalyst concentration at the outlet of the second magnetic separator 30 in TABLE 3.

TABLE 3

| | | EXAMPLE | EXAMPLE | EXAMPLE | EXAMPLE | EXAMPLE | COMPARATIVE |
|---|---|---|---|---|---|---|---|
| | | 10 | 11 | 12 | 13 | 14 | EXAMPLE 3 |
| PROCESS CONDITION | MAGNETIC FIELD | 30000 | 15000 | 15000 | 15000 | 15000 | FILTER |
| FOR FIRST HIGH | STRENGTH (Gauss) | | | | | | ELEMENT |
| GRADIENT | PROCESS | 150 | 150 | 150 | 150 | 150 | MESH |
| MAGNETIC | TEMPERATURE (° C.) | | | | | | OPENING |
| SEPARATOR | LIQUID RESIDENCE | 50 | 50 | 50 | 50 | 10 | SIZE: 10 μm |
| | TIME (SECOND) | | | | | | |
| PROCESS CONDITION | MAGNETIC FIELD | 15000 | 30000 | 15000 | 40000 | 20000 | |
| FOR SECOND HIGH | STRENGTH (Gauss) | | | | | | |
| GRADIENT | PROCESS | 150 | 150 | 150 | 150 | 150 | |
| MAGNETIC | TEMPERATURE (° C.) | | | | | | |
| SEPARATOR | LIQUID RESIDENCE | 50 | 75 | 150 | 60 | 150 | |
| | TIME (SECOND) | | | | | | |

| | | | | | | |
|---|---|---|---|---|---|---|
| MAGNETIC SUSCEPTIBILITY (emu/g) OF FT | 7.36 | 7.40 | 7.40 | 7.40 | 7.46 | 7.25 |
| CATALYST RECYCLED TO FT SYNTHESIS | | | | | | |
| REACTOR | | | | | | |
| CATALYST CONCENTRATION (mass ppm) AT | 9.6 | 9.7 | 9.7 | 8.6 | 8.8 | 10.0 |
| OUTLET OF SECOND MAGNETIC | | | | | | |
| SEPARATOR | | | | | | |

(Results)

Like Comparative Example 3, in the case where the FT catalyst particles removed by the filter are recycled, it is understood that even the catalyst having weak magnetism and low activity is recycled to the FT synthesis reactor. On the other hand, like the respective Examples, in the case where the FT catalyst particles separated by the first high gradient magnetic separator 20 and recycled to the FT synthesis reactor 10 have magnetism and catalytic activity higher than those of the FT catalyst particles contained in the slurry at the outlet of the FT synthesis reactor 10.

In any Examples in which the second high gradient magnetic separator 30 is used, it is understood that the FT catalyst concentration of the liquid B at the outlet of the second magnetic separator 30 is reduced to a value less than 10 mass ppm and the microparticle removal level is equal to or better than that of Comparative Example 3 in which only the filter is used.

### INDUSTRIAL APPLICABILITY

The present invention relates to a method of selectively removing the fine catalyst and the low-active catalyst from the Fischer-Tropsch synthetic crude oil and a method of recycling the high-active catalyst in the Fischer-Tropsch synthetic crude oil. According to the invention, it is possible to efficiently capture the fine catalyst from the FT synthetic oil in which fine microparticles are easily produced. Further, it is possible to selectively reuse the residual catalyst having strong magnetism and high activity.

1. A method of selectively removing a Fischer-Tropsch catalyst from a Fischer-Tropsch synthetic crude oil, the method comprising the steps of:
extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor;
separating a catalyst having a predetermined diameter or more from the slurry by means of a first solid-liquid separator; and
separating a catalyst which is not separated by means of the first solid-liquid separator from the slurry from which the catalyst having the predetermined diameter or more is removed, by means of a second solid-liquid separator,
wherein the catalyst separated from the slurry by means of the first solid-liquid separator is recycled to the Fischer-Tropsch synthesis reactor so as to be reused,
wherein the catalyst separated from the slurry by means of the second solid-liquid separator is discharged to the outside of a system, and
wherein an average particle diameter of the catalyst discharged to the outside of the system is smaller than that of the catalyst contained in the slurry at an outlet of the Fischer-Torpsch synthesis reactor.

2. The method according to claim 1, wherein the first solid-liquid separator is a high gradient magnetic separator, and
wherein the second solid-liquid separator is selected from a solid-liquid separator except for the high gradient magnetic separator.

3. The method according to claim 1, wherein the second solid-liquid separator is a high gradient magnetic separator, and
wherein the first solid-liquid separator is selected from a solid-liquid separator except for the high gradient magnetic separator.

4. The method according to claim 2 or 3, wherein the high gradient magnetic separator comprises a washing liquid introduction line for cleaning the inside thereof and a washing liquid discharge line for discharging washing liquid from the high gradient magnetic separator, and intermittently cleans magnetic particles captured inside of the high gradient magnetic separator.

5. The method according to claim 2 or 3, wherein the solid-liquid separator except for the high gradient magnetic separator is at least one of a filter, a gravitational sedimentation separator, a cyclone, and a centrifugal separator.

6. A method of selectively removing a Fischer-Tropsch catalyst from a Fischer-Tropsch synthetic crude oil, the method comprising the steps of:
extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor;
separating a catalyst having strong magnetism from the slurry by means of a first high gradient magnetic separator; and
separating a catalyst which has weak magnetism and is not be separated by means of the first high gradient magnetic separator from the slurry from which the catalyst is separated, by means of a filter,
wherein the catalyst which has strong magnetism and is separated from the slurry by means of the first high gradient magnetic separator is recycled to the Fischer-Tropsch synthesis reactor so as to be reused, and
wherein the catalyst which has weak magnetism and is separated from the slurry by means of the filter is discharged to the outside of a system.

7. The method according to claim 6, wherein magnetism of the catalyst separated from the slurry by means of the filter is weaker than that of the catalyst contained in the slurry at an outlet of the Fischer-Tropsch synthesis reactor.

8. A method of recycling a Fischer-Tropsch catalyst, the method comprising the steps of
extracting a slurry, containing a Fischer-Tropsch catalyst having magnetism and Fischer-Tropsch synthetic crude oil obtained by a Fischer-Tropsch synthesis reaction, from a Fischer-Tropsch synthesis reactor;
separating a catalyst having strong magnetism from the slurry by means of a first high gradient magnetic separator; and
separating a catalyst which is not be separated by the first high gradient magnetic separator from the slurry from which the catalyst having strong magnetism is separated, by means of a second high gradient magnetic separator,
wherein the catalyst which has strong magnetism and is separated from the slurry by means of the first high gradient magnetic separator is recycled to the Fischer-Tropsch synthesis reactor so as to be reused, and
wherein the catalyst separated from the slurry by means of the second high gradient magnetic separator is discharged to the outside of a system.

9. The method according to claim 8, wherein magnetism of the catalyst separated from the slurry by means of the first high gradient magnetic separator is stronger than that of the catalyst contained in the slurry at an outlet of the Fischer-Tropsch synthesis reactor.
